(19) United States
(12) Patent Application Publication
(10) Pub. No.: US 2008/0050457 A1
(43) Pub. Date: Feb. 28, 2008

(54) Ginkgo Biloba Extract And Method For Producing The Same

(76) Inventor: Fu Yuanhui, Beijing (CN)

Correspondence Address: SCHWEITZER CORNMAN GROSS & BONDELL LLP, 292 MADISON AVENUE - 19th FLOOR, NEW YORK, NY, 10017, US

(21) Appl. No.: 11/587,721

(22) PCT Filed: Apr. 29, 2005
(86) PCT No.: PCT/EP05/04631

§ 371 Date: Sep. 21, 2007

(30) Foreign Application Priority Data
Apr. 29, 2004 (DE) 10 2004 021 276.7

(51) Int. Cl. A61K 36/16; A61P 25/00; A61P 43/00; A61P 9/00

(52) U.S. Cl. 424/752

(57) ABSTRACT

The invention relates to a Ginkgo biloba extract and to a method for producing the same. The invention especially relates to a Ginkgo extract having an increased content in desired, pharmaceutically effective active ingredients and to a method which uses water as the extractant.

The invention relates to a Ginkgo biloba extract and method for producing the same. In particular, the invention relates to a Ginkgo extract having an increased content of desired pharmaceutically active ingredients, and methods which exclusively make use of water and alcohol, respectively, as a medium for extraction.

Extracts from leaves of the gingko biloba tree (Ginkgo biloba L.) and pharmaceutical compositions prepared therefrom are subject to extensive pharmacological studies and are used in the medical and cosmetic field. In the medical field Ginkgo extracts are particularly useful in the treatment of circulatory insufficiency and vascular disorders, in the treatment of disorders caused by cerebral-organic dysfunction such as memory disturbance and lack of concentration, headache and noises in the ear.

Studies revealed that leaves of Ginkgo contain more than 70 different ingredients such as flavon glycosides, quercetin, isorhamnetin, kaempferol, ginkgetin, isoginkgetin, ginkgolides A, B, C and J, biolobalide and others. The pharmaceutical effect of Ginkgo extracts is mainly described to the ingredients Ginkgol flavon glycosides, ginkgolides and bilobalides. The mentioned individual ingredients show a similar pharmaceutical effect as Ginkgo leaves or extracts made from said leaves, the is efficacy is, however, lower than that of the leaves or the extracts. This is presumably attributable to a synergistic effect of the ingredients. It was also conceivable that some of the active ingredients contained in the leaves have not yet been identified.

Among the desired pharmaceutically active ingredients Ginkgo leaves also contain contents of undesired ingredients which may cause allergic reactions. Allergic reactions after having been in contact with Ginkgo leaves and Ginkgo fruits have been reported (see W. F. Sowers et al., Arch. Dermatol., Vol. 91 (1965), 452). As theses allergic reactions causing substances alkyl phenolic compounds such as Ginkgolic acids and Ginkgols, proanthocyanidines and resorcine derivatives thereof have been identified (see J. Gellermann et al., Phytochemistry, Vol. 15 (1976), 1959).

For obtaining the desired pharmaceutical activity Ginkgo leaves might of course be used directly. However, this has the disadvantage that undesired allergenic ingredients would be administered. Moreover, the contents of ingredients in the leaves may vary significantly so that no targeted dosing might be possible for therapeutic use.

As a solution to this problem, the use of Ginkgo extracts with a relatively constant active ingredient profile has been suggested. Many suggestions have been made in the state of the art for suitable extraction methods and for the preparation of extracts with defined active ingredient profiles.

DE 21 17 429 suggests a method for preparing extracts from Ginkgo leaves, whereby leaves of Ginkgo are extracted with aqueous acetone, the obtained extract is subjected to an extraction with carbon tetrachloride, butanone and ethanol, and the obtained solution was then subjected to a precipitation reaction with lead hydroxide. Upon further method steps, an extract is finally obtained.

EP 0 431 535 discloses a method in which dried leaves of Ginkgo biloba are extracted with aqueous acetone, an aqueous alkanol or anhydrous methanol, and whereby in further method steps additional organic solvents, such as methylethylketone, are used.

International patent application WO 00/01397 discloses a method of extraction, in which Ginkgo leaves are extracted with alcoholic-aqueous or ketonic-aqueous solvents and the obtained extracts are subsequently subjected to ultrafiltration.

The extraction methods described in the state of the art have the disadvantage that pollutive and physiologically unacceptable organic solvents are used which, on the one hand, may contaminate the obtained extract and which use, on the other hand may have disadvantages under environmental policy, safety policy and last but not least economical aspects. Moreover, the use of heavy metal compounds as precipitating agents as suggested in the state of the art has disadvantages in that the obtained extracts may be contaminated with heavy metals.

Furthermore, the methods known in the art have the disadvantage that they result in extracts with a relatively low content of desired ingredients. For example, EP 0 431 535 describes an extract, which contains almost 30 wt.-% of flavon glycosides, 4.5 wt.-% of gingkolides and at most 4 wt.-% of bilobalides at most, wherein the content of undesired alkyl phenolic compounds is lower than 10 ppm. This substance profile suffices the requirements of the Monograph for “Ginkgo biloba Trockenextrakt” of the commission E of the previous Federal Public Health Department of the Federal Republic of Germany.

In international patent application WO 2004/014405 a Ginkgo extract is described which contains 5.5-8% Ginkgolides A, B, C and J, 40-60% flavonglycosides and 5-7% bilobalide. No information is given about the content of alkyl phenolic compounds and proanthocyanidines, and to the process for the production of the extract, respectively.

It is an object of the present invention to provide a method for preparing extracts from leaves of Ginkgo biloba which does not make use of environmentally harmful or physiologically unacceptable organic solvents.

It is a further object of the invention to provide a Ginkgo biloba extract which is not contaminated with organic solvents and/or heavy metals.

It is a further object of the present invention to provide a Ginkgo biloba extract having an increased content of desired ingredients, i.e. flavonglycosides, Ginkgolides and/or bilobalide, having at the same time toxicologically harmless low contents of allergenic ingredients.

These objects are attained by the features according to the independent claims.

The dependent claims define preferred embodiments of the methods and extracts of the present invention.

It has been surprisingly found in the studies underlying the present invention that Ginkgo extracts having a substance profile as required by the Monograph are obtainable by a method in which exclusively water is used as a solvent. This method substantially comprises the following steps:
1. Treatment of Ginkgo leaves, preferably leaves derived from biological cultivation, with an alkaline aqueous solution, preferably saturated lime water. Due to this treatment step, in which Ginkgo leaves are dipped into an alkaline aqueous solution, impurities on the surface of the leaves are removed and the leaves' wax layer are destroyed. Thereby, the leaves are re-hydrated, i.e. the cells of the leaves absorb water and swell. Preferably, the leaves are dipped into water having a pH value of about 8.5 for a time period of about 4 hours. Subsequently, the water used for the treatment is dumped and the remaining leaves are rinsed with water.
2. The leaves obtained in step 1 are extracted with water, which may be tapwater but is preferably demineralized or distilled water. The amount of water used is about 5 to 20 times, preferably 5 to 15 times and most preferably 8 to 10 times of the dry weight of the leaves. The extraction may be conducted at room temperature or slightly elevated temperatures between 40 and 70° C., whereby lower temperatures are preferred in order to exclude hydrolysis.
   The extraction may be conducted using common procedures such as simply dipping, diacolation, ultrasonic extraction or mechanical stirring. The extraction is performed over a time period of 0.5 to 5 hours depending on the extraction procedure chosen.
3. The obtained extract is then filtered using common procedures and materials in order to remove larger particles. Filter paper or fabrics, diatomite or the like, may be used. Filtration can be conducted under normal pressure, elevated pressure or below atmospheric pressure.
   The filtered extract is then subjected to ultrasonic filtration using several common ultrafiltration membranes. Preferably the extract is first filtered through a membrane having an average pore size of about 10,000 Daltons, subsequently through a membrane having a pore size of about 6,000 Daltons and finally over a membrane having a pore size of about 2,000 Daltons. Filters made from polyamide, polypropylene or other common materials are used.
   Upon ultrafiltration larger molecules such as wax components, proteines, tannines or polysaccharides, are separated first by means of the coarse membranes, and smaller molecules, such as proteines, starches or oligosaccharides, are separated by means of the finer membranes.
4. The extract obtained after ultrafiltration is then subjected to reverse osmosis, whereby commonly used semipermeable membranes and devices for reverse osmosis are used. For example, the reverse osmosis can be conducted by means of a celluloseacetate membrane at a pressure of, e.g. 4.5 MPa, whereby the pressure is adapted to the respective needs. Using reverse osmosis, heavy metal ions and smaller sugar molecules are separated.
5. The extract obtained after ultrafiltration is then given onto a chromatographic absorption column. The column is filled with an absorption material which can absorb allergenic substances, such as Ginkgolic acid, and which does not react with the desired ingredients of the extract. As an immobile phase in particular large absorption porous resins, or active carbon may be used. It was found in the present invention that carbon derived from nutshells of Ginkgo (active carbon derived from carbonized nutshells of Ginkgo) is a suitable absorption material. Particularly preferred resins are those available under the trademark GARA 1 and GARA 2 from the company Woxun Bioengineering Ltd., Shanghai, China, and Mitsubishi SP700, respectively.

Extracts obtained with the method described above may be dried using common procedures, e.g. by distillation under optionally reduced pressure or lyophilization. The extracts obtained in such a way show a substance profile which complies with the requirements of the Monograph “Ginkgo biloba Trockenextrakt” of commission E of the previous Federal Public Health Department of the Federal Republic of Germany. They typically contain 24-27 wt.-% flavonglycosides, 3.5-4 wt.-% Ginkgolides and 2.4-2.6 wt.-% bilobalide, less than 10 ppm, even less than 2 ppm alkyl phenolic compounds, and less than 10 wt.-% proanthocyanidines. The extracts have a content of heavy metals of less than 0.12 mg/kg lead, less than 0.1 mg/kg cadmium and less than 0.08 mg/kg mercury. The solubility in water of the dry extracts obtained using the procedure described above is more than 1.2 g/100 ml water.

According to the present invention contents of flavonglycosides, Ginkgolides and bilobalide in the extracts can be significantly increased, if an additional method step is carried out.

The aqueous extract obtained in above step 5 may additionally be given onto a chromatographic column that contains as a stationary phase an absorption resin, preferably on the basis of a crosslinked polystyrol, preferably those resins available under the tradename Diaion® and Sepabeads® from Mitsubishi, more preferably Mitsubishi Diaion HP20. Upon rinsing the loaded resins with water, subsequently rinsing with an alcoholic-aqueous solution, and finally eluting the extract containing the desired ingredients, extracts having variable substance profiles may be obtained. Rinsing with alcoholic-aqueous solution can be conducted several times with increasing concentrations of alcohol, whereby the alcohol concentration is preferably between 10 and 25%. For example, the washing can be conducted in a first step with 10% alcohol and in a second step with 20% alcohol in order to separate further undesired ingredients. The alcoholic-aqueous solution used for rinsing contains up to 25%, preferably 10-20% of a lower molecular alcohol such as isopropanol, methanol or ethanol, wherein ethanol being particularly preferred. The solutions obtained after rinsing are thrown away; for the subsequent elution of the extract an aqueous-alcoholic solution is used which contains at least 75% of one of the above described alcohols, preferably ethanol. Depending on the chosen conditions extracts having the following substance profiles may be obtained:

| | | |
|---|---|---|
| flavonglycosides | 8.0-52 | wt.-% |
| gingkolides A, B, C, J | 2.8-41 | wt.-% |
| bilobalide | 2.6-39 | wt.-% |
| proanthocyanidines | 1-20 | wt.-% |
| alkyl phenols | less than 2.0 | ppm |
| tannines | less than 5 | wt.-%. |

All indications of weight relate to the weight of the obtained dry extract.

By using the process of the present invention it is possible to obtain Ginkgo biloba extracts having significantly higher contents of desired ingredients (flavonglycosides, Ginkgolides and bilobalides) compared to extracts known in the art, whereby the contents of undesired allergenic substances are not significantly increased and the requirements of the Monograph are complied with.

The following examples further illustrate the present invention.

### EXAMPLE 1

50 kg dried Ginkgo leaves were dipped into saturated lime water having a pH value of 9.5 at ambient temperature until the leaves were saturated with water. The lime water was poured off and thrown away. The remaining leaves were then washed with pure water and pounded. Subsequently, they were treated with 450 kg dionisized water and extracted for 1.5 hours using ultrasonic. The water temperature decreases during the treatment with ultrasonic and was kept at 45 to at most 48 C.°.

After having finalized the extraction, coarse-particulated residues were separated by filtration. The obtained extract was then filtrated using a polypropylene ultrafiltration membrane having a pore size of 10,000 Daltons, followed by a ultrafiltration membrane having a pore size of 6,000 Daltons and finally a ultrafiltration membrane having a pore size of 2,000 Daltons. The filtrate finally obtained was then subjected to a reverse osmosis treatment using a cellulose acetate membrane. The salt permeation rate was at least 95% by weight, the water permeation rate was 90-1201/m² h at 4.5 MPa.

The filtrate obtained in such a way was then given onto a chromatographic column having a diameter of 30 cm and a height of 1.2 m packed with carbonized nutshells of Ginkgo. Chromatographic separation was carried out at ambient pressure and temperature.

The eluent was concentrated to a mass of less than 8 kg by using the above described reverse osmosis membrane and finally spray dried to obtain a 1.4 kg extract.

The obtained extract was analyzed by means of HPLC. The total content of flavones was 26.7 wt.-%., the content of Ginkgolides A, B and C was 3.54 wt.-%, the content of bilobalide was 2.58 wt.-%, the content of Ginkgolic acids 1 (a and b) was 0.39 and 3.37 ppm, respectively, the content of Ginkgolic acid 2 was less than 0.3 ppm and the content of heavy metals was less than 0.12 mg/kg lead, less than 0.1 mg/kg cadmium, and less than 0.08 mg/kg mercury. (The weight percent ages again relate to the mass of the obtained dry extract).

### EXAMPLE 2

1 kg dried leaves of Ginkgo were dipped into 8 kg water, saturated with CaO (approximately 120 g CaO per litre of water), and kept at ambient temperature for half an hour. Subsequently, the water was poured off and the moist leaves were washed with dionized water. Then one litre water was added per 100 g leaves and the batch was treated in a ultrasonic bath for about 45 minutes (approximately 20-24 kHz). The aqueous extract obtained was separated and stored. After adding one additional litre water per 100 g leaves the treatment with ultrasonic was repeated, and the resulting aqueous extract was combined with the extract obtained before.

The combined extracts were then centrifugated at 15.000 rpm and the solids and oil components were separated from the extract.

The obtained extract was then filtrated using a Millipore Labscale TTF Pellicon XL ultrafiltration system, wherein ultra filtration membranes with pore sizes of 10,000 Daltons, 8,000 Daltons and 5,000 Daltons were used. Subsequently, a filtration was conducted by means of membranes having a pore size of 3,000 Daltons and 1,000 Daltons. The filtrate obtained was then subjected to a reverse osmosis treatment using a cellulose acetate membrane. The salt permeation rate was at least 95 wt.-%, the water permeation rate was 90-120 l/m² h at 4.5 MPa.

For removing Ginkgolic acids the extract obtained was given onto a column packed with the resin G 1.5 g of the resin were used for 100 ml extract. The chromatographic separation was conducted at ambient pressure.

For removing proanthocyanidine the liquid obtained in the previous step was given onto a column that was packed with the resin Diaion HP2MGL. 5 g of the resin were used per 100 ml of the liquid. The chromatography was conducted at ambient temperature and ambient pressure.

The obtained liquid was then given onto a column packed with the resin Mitsubishi Diaion HP20; 2 g of the resin were used per 100 ml of the liquid. As soon as some tea-coloured contents left the column the procedure was stopped. Subsequently, the column was washed with pure water and then with 10% aqueous-ethanolic solution. The washing solutions were thrown away. Subsequently, the column was eluted with 75% ethanolic aqueous solution, and the liquid obtained was collected. The obtained liquid was dried at low temperature to afford the desired extract.

Under variation of the concentration of the ethanolic-aqueous solution used for washing the column, extracts with following substance profiles were obtained:

a) Composition of the extract:

| | | |
|---|---|---|
| flavonglycosides | 39 | wt.-% |
| ginkgolides A, B, C and J | 2.9 | wt.-% |
| bilobalide | 2.78 | wt.-% |
| proanthocyanidines | 10.5 | wt.-% |
| alkyl phenoles | <0.7 | ppm |
| tannines | <5 | wt.-% |

b) Composition of the extract:

| | | |
|---|---|---|
| flavonglycosides | 35 | wt.-% |
| ginkgolides A, B, C und J | 6.67 | wt.-% |
| bilobalide | 6.49 | wt.-% |
| proanthocyanidines | 14 | wt.-% |
| alkyl phenoles | <0.70 | ppm |
| tannines | <5 | wt.-% |

c) Composition of the extract:

| | | |
|---|---|---|
| flavonglycosides | 31 | wt.-% |
| ginkgolides A, B, C und J | 10 | wt.-% |
| bilobalide | 9 | wt.-% |
| proanthocyanidines | 11 | wt.-% |
| alkyl phenoles | <0.70 | ppm |
| tannines | <5 | wt.-% |

d) Composition of the extract:

| | | |
|---|---|---|
| flavonglycosides | 18 | wt.-% |
| ginkgolides A, B, C und J | 21.23 | wt.-% |
| bilobalide | 20.17 | wt.-% |
| proanthocyanidines | 14 | wt.-% |
| alkylphenoles | <0.70 | ppm |
| tannines | <5 | wt.-% |

e) Composition of the extract:

| | | |
|---|---|---|
| flavonglycosides | 49 | wt.-% |
| ginkgolides A, B, C und J | 3 | wt.-% |
| bilobalide | 2.7 | wt.-% |
| proanthocyanidines | <2 | wt.-% |
| alkyl phenoles | <3 | ppm |
| tannines | <1 | wt.-% |

f) Composition of the extract:

| | | |
|---|---|---|
| flavonglycosides | 51.5 | wt.-% |
| ginkgolides A, B, C und J | 2.8 | wt.-% |
| bilobalide | 2.6 | wt.-% |
| proanthocyanidines | <2 | wt.-% |
| alkyl phenoles | <3 | ppm |
| tannines | <1 | wt.-% |

(all wt.-% values relate to the mass of the dry extract)

The examples show that by using the method of the present invention dried extracts of Ginkgo biloba leaves are obtained, which have significantly increased contents of desired ingredients, at the same time having sufficiently low contents of undesired allergenic ingredients.

The extracts of the present invention allow the production of pharmaceutical and cosmetic compositions having increased contents of active ingredients. They may be manufactured to generally used pharmaceutical compositions, preferably tablets, solutions for injection and solutions for oral application, without any problems.

### EXAMPLE 3

By using the extracts of examples 1 and 2 solutions for oral application having the following composition were prepared:

| | | |
|---|---|---|
| Ginkgo biloba extract | 3.0 | g |
| ethanol | 50.0 | g |
| water for injection | ad 100 | ml |

### EXAMPLE 4

By using the extracts of examples 1 and 2 tablets having the following composition were prepared:

| | | |
|---|---|---|
| Ginkgo biloba extract | 30.00 | mg |
| microcrystalline cellulose | 100.00 | mg |
| lactose | 60.00 | mg |
| colloidal silica | 20.00 | mg |
| magnesium stearate | 3.00 | mg |
| HPMC | 10.00 | mg |
| talc | 0.5 | mg |

1. Extract from Ginkgo leaves biloba which contains more than 8 wt.-% Ginkgolides A, B, C and J (total) and/or more than 7 wt.-% bilobalide.

2. Extract according to claim 1 which contains less than 10 ppm alkyl-phenolic compounds and less than 20 wt.-% proanthocyanidines.

3. Extract according to claim 1 or 2 which contains
more than 30 wt.-% up to 60 wt.-% of flavonglycosides,
more than 8 wt.-% up to 40 wt.-% Ginkgolides A, B, C and J (total),
more than 7 wt.-% up to 35 wt.-% bilobalide,
less than 10 ppm alkyl-phenolic compounds and
less than 20 wt.-% proanthocyanidines.

4. Method of preparation of an extract from Ginkgo biloba leaves comprising the following steps:
treating leaves of Ginkgo biloba with water having a pH value of more than about 8.5 until the leaves are saturated with water;
extraction of the obtained leaves with water;
filtration of the obtained extract;
chromatographic purification of the obtained extract with a coarse porous absorption resin or active carbon; and
chromatographic purification of the extract obtained in the previous step with an absorption resin.

5. Method according to claim 4, characterized in that the extraction is carried out with lime water.

6. Method according to claim 4 or 5, characterized in that the filtration step comprises ultrafiltration.

7. Method according to claim 6 characterized in that the ultrafiltration is carried out with membranes having pore sizes of 10,000 Daltons, 6,000 Daltons and 2,000 Daltons.

8. Method according to any one of claims 4-7, characterized in that the final chromatographic purification comprises rinsing with up to 25% alcoholic-aqueous solution and eluting the extract with an at least 75% alcoholic-aqueous solution.

9. Method according to claim 8, characterized in that ethanol is used as an alcohol.

10. Pharmaceutical composition comprising a content of Ginkgo biloba extract according to any one of claims 1-8.
